(19) United States
(12) Patent Application Publication
(10) Pub. No.: US 2026/0026744 A1
(43) Pub. Date: Jan. 29, 2026

(54) MICRONEEDLE ARRAY SYSTEMS AND DEVICES, AND METHODS ASSOCIATED WITH MANUFACTURING AND USE

(71) Applicant: Panther Life Sciences Corporation, Normandy Park, WA (US)

(72) Inventors: Ralph SMITH, St. Paul, MN (US); Brian ROGERS, Kent, CT (US); Carlos BHOLA, Seattle, WA (US); Jorge E. SALAZAR, New York, NY (US)

(21) Appl. No.: 19/290,146

(22) Filed: Aug. 4, 2025

Related U.S. Application Data
(63) Continuation of application No. PCT/US2025/039322, filed on Jul. 25, 2025.
(60) Provisional application No. 63/675,692, filed on Jul. 25, 2024.

(51) Int. Cl. A61B 5/00; G16H 10/60; G16H 20/17; G16H 20/40

(52) U.S. Cl. CPC A61B 5/4833; A61B 5/4839; A61B 5/685; G16H 10/60; G16H 20/17; G16H 20/40; A61B 2562/08

(57) ABSTRACT

The present disclosure relates to microneedle array patches, and systems and devices including the same. Each microneedle array patch can include a plurality of microneedles including an agent that is loaded into at least a tip of the microneedles, a base structure configured to support the plurality of microneedles when force is applied against the second flat surface to insert the plurality of microneedles into skin; and an adhesive disposed around the plurality of microneedles, the adhesive configured to secure the base structure and the plurality of microneedles to the skin for a predetermined period of time. The microneedle array patches can include identifiers and/or sensors for measuring information regarding the microneedle array patches and/or retrieving information regarding the microneedle array patches.

### CROSS-REFERENCE TO RELATED APPLICATIONS

This application is a continuation of PCT Application No. PCT/US2025/039322, filed Jul. 25, 2025, which claims priority to and the benefit of U.S. Provisional Patent Application No. 63/675,692, filed Jul. 25, 2024, the disclosure of each of which is incorporated herein by reference.

### FIELD OF THE DISCLOSURE

This disclosure relates to systems and devices including microneedle arrays, and methods thereof.

### BACKGROUND

A variety of systems and devices including microneedle arrays have been developed for intradermal and/or transdermal delivery of agents. Such microneedle arrays can be configured to piece through layers of skin to deliver agents to the stratum corneum and/or dermis and/or epidermis of a subject. It can be desirable to monitor and/or manage the manufacture and use of such microneedle arrays.

The effective management and tracking of medical objects, including drugs, treatments, drug delivery devices, and medical equipment, throughout their lifecycle is critical for ensuring safety, efficacy, and compliance within the healthcare industry. Traditionally, tracking these objects has involved a combination of manual record-keeping, barcode systems, and isolated databases, leading to inefficiencies and potential errors.

Existing systems often lack a cohesive approach that integrates multiple stages of the medical object's journey—e.g., from manufacturing to end-use—resulting in fragmented data that can hinder timely decision-making and compromise patient safety. Furthermore, manual and semi-automated tracking methods are prone to human error, leading to inaccurate records and potential misuse of medical objects.

Despite advancements in technology, there remains a gap in providing a seamless, end-to-end tracking solution that not only monitors the movement of medical objects through the supply chain but also captures critical patient data before, during, and after treatment. This data is vital for ensuring that treatments are administered correctly, monitoring patient outcomes and discovering long-term efficacy. Current systems fail to effectively integrate and manage this information in a comprehensive and accessible manner.

Additionally, existing solutions do not adequately address the diverse roles and needs of different system users—e.g., patients, clinicians, and manufacturers. Patients require easy access to treatment information, clinicians need reliable data to make informed decisions, and manufacturers must ensure compliance and traceability of their products. The lack of a unified system that caters to these distinct user groups further complicates the effective management of medical objects.

### SUMMARY OF THE DISCLOSURE

The present disclosure relates to microneedle arrays, and systems, devices, and methods thereof. In some embodiments, systems and devices described herein include microneedle arrays that are configured for intradermal and/or transdermal delivery of one or more agents.

In some embodiments, a system, comprising: a microneedle array patch including: a base structure including first and second flat surfaces; a plurality of microneedles disposed on the first flat surface of the base structure, the plurality of microneedles configured to extend out from the first flat surface, each microneedle of the plurality of microneedles including an agent that is loaded into at least a tip of the microneedle, the base structure configured to support the plurality of microneedles when force is applied against the second flat surface to insert the plurality of microneedles into skin; and an adhesive disposed around the plurality of microneedles, the adhesive configured to secure the base structure and the plurality of microneedles to the skin for a predetermined period of time, an identifier disposed on the microneedle array patch; a sensor configured to read the identifier; and a processor operative coupled to the sensor, the processor configured to: receive, from the sensor, the identifier of the microneedle array patch, wherein the identifier is associated with an identifier record; query an information system to confirm the identifier and the microneedle array patch associated with the identifier are valid; in response to receiving a response from the information system indicating that the identifier and the microneedle array patch are valid, present instructions to the user to administer the microneedle array patch; and in response to receiving a response from the informing system indicating that the identifier and the microneedle array patch are not valid, present an alert to the user.

In some embodiments, a method includes: generating an identifier record for an identifier affixed to a microneedle array patch, the identifier being affixed to the microneedle array patch during manufacturing of the microneedle array patch, the microneedle array patch including a base structure and a plurality of microneedles disposed on a flat surface of the base structure, the base structure configured to provide support to the plurality of needles during insertion of the plurality of microneedles into skin; generating an object record for the microneedle array patch; associating, by the information system, the identifier record with the object record so that the microneedle array patch including the identifier affixed thereon is associated with the identifier; in response to receiving prescription information for a patient from a first device associated with a prescribing entity, associating a patient record of the patient with the identifier record so that the microneedle array patch including the identifier affixed thereon is associated with the patient; receiving a request to validate the identifier and information of the patient from a second device associated with the patient prior to administration of the microneedle array patch; validating the identifier and the information of the patient based on the object record, the identifier record, and the patient record and the associations therebetween, and sending a response to the second device based on the validation; and sending, to a third device associated with managing electronic records of patients, treatment data associated with the administration of the microneedle array patch such that the third device can update electronic records of the patient.

In some embodiments, a method includes: receiving, by a processor, an identifier of a microneedle array patch from a sensor, wherein the identifier is associated with an identifier record, wherein the microneedle array patch includes a base structure and a plurality of microneedles disposed on a flat surface of the base structure, the base structure configured to provide support to the plurality of needles during insertion of the plurality of microneedles into skin; querying an information system to confirm the identifier and the microneedle array patch are valid; in response to receiving a response from the information system indicating that the identifier and the microneedle array patch are valid, present instructions to the user to administer the microneedle array patch; and in response to receiving a response from the informing system indicating that the identifier and the microneedle array patch are not valid, present an alert to the user.

In some embodiment, the present disclosure involves systems and methods for tracking medical objects, such as, for example, microneedle arrays, drugs, treatments, drug delivery devices, and/or medical equipment, from manufacture to use. In some embodiments, the system can include a networked information system, software applications, and a unique identifier added to each medical object during manufacturing. This identifier is associated with object data in the information system. The information system tracks the medical object from manufacturing, through the supply chain, to the patient, capturing patient vitals and usage data before, during, and after treatment.

In some embodiments, the disclosure can involve a networked information system, software applications, and unique identifiers. During manufacturing, a unique identifier—which may be, for example, encoded in a quick-response (QR) code, bar code, radiofrequency identification (RFID) chip, serial number, or other—is added to a medical object (e.g., drug, treatment, drug delivery device, medical equipment). The information system creates a record associating the identifier with the object and tracks it as it moves through the supply chain from manufacturing to the patient.

In some embodiments, the information system is a networked application incorporating a database. It can be queried via an Application Programming Interface (API) and stores information used by users such as patients, clinicians, and manufacturers.

Patients may use a software application to facilitate their treatment. The application, when implemented by a processor, can be configured to read the unique treatment identifier using sensors, such as, for example, a camera or radiofrequency antenna for Near Field Communication (NFC) or Bluetooth. The application checks the information system's association between the user and the object and retrieves object information. The patient application provides users treatment information such as, for example, instructions. expiration dates, disclosures, and recall notifications.

The patient application may record the patient's use of the device and any associated metadata, including, for example, the location, time, and health vitals before, during, and after treatment. Heart rate, blood pressure, temperature, skin conductance, EKG, and blood sugar levels can be captured. e.g., via wearable electronic sensors, smartphones, smart devices, or embedded electronics in the identifier or the medical object itself. The patient application uploads the treatment data and metadata to the information system. Where permitted, manufacturers and clinicians can have access to patient data to facilitate care and research.

Patients benefit from accessing comprehensive information about their treatments and usage, enhancing their knowledge and safety. This transparency empowers patients, allowing them to make informed decisions about their healthcare and fostering greater trust in the treatment process, leading to better patient outcomes.

Furthermore, the system can issue real-time recall notifications, preventing unnecessary harm to patients by quickly removing defective or potentially harmful medical objects from circulation. By verifying medical objects before use, the system ensures that each patient receives the correct treatment, thereby decreasing medical errors and enhancing patient safety.

In some embodiments, a clinician application, when implemented by a processor, allows clinicians to track their patients' treatments, monitor adherence to treatment protocols, assess treatment effects, and identify adverse events. For clinicians, the system provides real-time visibility and tracking of adverse patient events, enabling a prompt response. Doctors can monitor patient compliance with treatment protocols, allowing for timely corrective actions and patient education, leading to better patient outcomes. Proactive monitoring can be crucial for managing chronic conditions and ensuring effective treatment.

In some embodiments, a manufacturing application, when implemented by a processor, enables the drug developer and manufacturer to track the number of treatments required, manufactured, shipped, and used. It may provide detailed information for business analytics. Data on patient treatments can be used for post-marketing surveillance studies, evaluating long-term safety and effectiveness, exploring new indications, updating product expirations, and identifying drug interactions.

The present disclosure offers a robust and integrated solution for tracking medical objects throughout their lifecycle. It addresses the needs of patients, clinicians, and manufacturers, significantly improving medical treatment management, safety, and effectiveness.

### BRIEF DESCRIPTION OF THE DRAWINGS

Some embodiments of the present disclosure are depicted for illustrative purposes and are not constrained by the figures of the accompanying drawings. In these drawings, reference numerals may denote similar elements across the various views.

FIG. 1 illustrate an anatomy of the skin with a microneedle array placed therein, according to embodiments.

FIG. 2 schematically depicts a microneedle array patch including a microneedle array, according to embodiments.

FIG. 3 depicts an isometric view of one example of a microneedle array, according to embodiments.

FIG. 4A shows an example of a system that interfaces with manufacturing, patient, and clinician applications to track a medical object such as a microneedle array, according to embodiments.

FIG. 4B is a schematic diagram showing a system for tracking information corresponding to performance and use of an MNA patch, according to embodiments.

FIG. 5 shows software layers of a system for tracking information regarding a medical object such as a microneedle array, according to embodiments.

FIG. 6 shows a flow chart of a method of tracking treatments from manufacturing to use of a medical object such as a microneedle array, according to embodiments.

FIGS. 7A-7C depict flow charts of associating a medical object such as a microneedle array with a unique identifier, according to embodiments.

FIG. 8 is a graphical representation of an identifier being affixed to a medical object such as a microneedle array, according to embodiments.

FIG. 9 shows a sensor configured to read an identifier of a medical object such as a microneedle array for pairing an identifier with the object, according to embodiments.

FIG. 10 shows a flow chart of a treatment flow for a patient treatment application that is used with a medical object such as a microneedle array, according to embodiments.

FIG. 11 shows an example device that can implement a patient treatment application and can interface with an identifier of a medical object and an information system, according to embodiments.

FIG. 12 shows a system for tracking an individual medical object from manufacturing to use, according to embodiments.

FIG. 13 shows a flow chart of method of recalling a medical object such as a microneedle array, according to embodiments.

### DETAILED DESCRIPTION OF THE DISCLOSURE

Different embodiments of systems, devices, and methods involving microneedle arrays are described herein. In some embodiments, systems, devices, and methods can include information system, user applications, and identifiers are discussed in detail herein.

Absorbable and/or degradable microneedles can be formed from or include biocompatible, absorbable and/or dissolvable polymers. As described herein, the microneedles arrays are configured to deliver agents (e.g., therapeutic, bioactive, non-active, etc.) to a subject. Absorbable microneedles are typically structured in an array referred to herein as an absorbable microneedle array or MNA. Agents may be embedded within the structural polymer and/or premixed with the structural polymer. A single absorbable MNA may have one agent or multiple agents embedded and/or premixed (“loaded”) with the structural polymer. A MNA may be designed with specific characteristics (e.g., microneedle geometry, tip sharpness, needle spacing) to (i) penetrate the stratum corneum without mechanical failure (e.g., needle fracture, needle deformation, etc.); (ii) target and reach the epidermis and/or dermis of the skin, which is approximately 50 to approximately 750 microns below the stratum corneum; and (iii) rapidly dissolve and/or absorb into the skin (e.g., via needle polymer) to deliver embedded and/or premixed API. In some embodiments, an absorbable MNA may include an array of needles having a stem structure and/or tip structure with specific geometry, e.g., to improve skin penetration, mechanical strength, etc. In some embodiments, an API can be loaded into and/or exclusively subside in a tip of the MNA. Further details of example MNAs are provided in U.S. Provisional Patent Application No. 63/675,689, entitled, “MICRONEEDLE ARRAYS WITH HETEROGENEOUS NEEDLES. AND SYSTEMS AND COMPONENTS THEREOF.” filed Jul. 25, 2024, and U.S. Pat. No. 9,944,019, entitled “TIP-LOADED MICRONEEDLE ARRAYS FOR TRANSDERMAL INSERTION,” published Apr. 17, 2018, which are both incorporated herein by reference in their entireties.

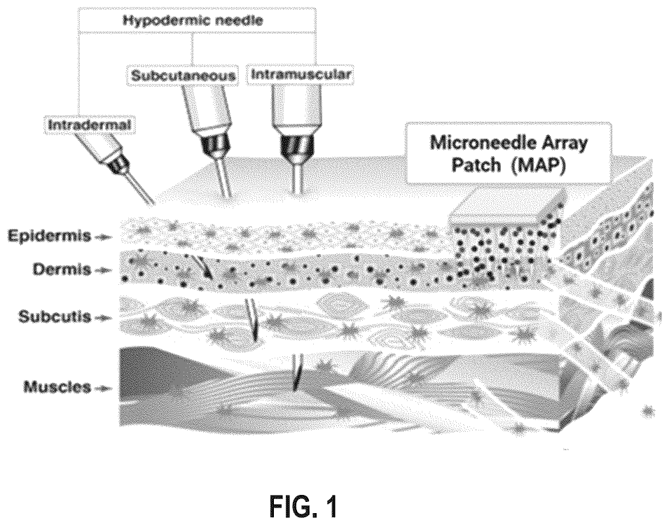

As described herein, the microneedles of the microneedle array can be configured to penetrate through and/or into layers of tissue to deliver the agents into the dermis and/or epidermis of a subject, depending on the desired biological compartment for agent delivery. FIG. 1 depicts a microneedle array, where needles of the microneedle array have been inserted into tissue, e.g., to deliver agents into the dermis and/or epidermis of a subject. In some embodiments, the microneedles of the microneedle array are configured to penetrate into the upper layers of the dermis to deliver an agent.

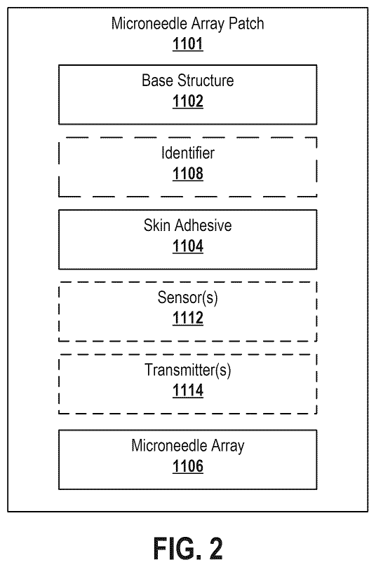

FIG. 2 depicts an example of a microneedle array patch or MNA patch 1101, according to embodiments. The MNA patch 1101 can include a microneedle array 1106, a base structure 1102 (e.g., base plate), and a skin adhesive 1104. Optionally, the MNA patch 1101 can also include an identifier 1108, one or more sensors 1112, and/or one or more transmitters 1114 configured to send the sensor data from the one or more sensors 1112 to a processor of an external device (e.g., an information system and/or a third-party device such as a compute device associated with manufacturing. a compute device associated with a prescriptive entity, a user device, a compute device associated with a pharmaceutical company, etc.). The microneedle array 1106 may include a first surface including a plurality of absorbable needles and a second surface opposite the first surface that is configured to be coupled to the skin adhesive 1104 and/or the base structure 1102. In some embodiments, the second surface opposite the first surface may be substantially flat, non-conformable, and composed of non-dissolving and/or absorbable material. In some embodiments, the second surface opposite the first surface may be substantially flat, conformable, and composed of the same material as the first surface and/or absorbable needles. In some embodiments, the microneedle array 1106 may be coupled to the base structure 1102 via any suitable mechanism (e.g., built-in snapfits, friction fit, adhesive, clips, magnets, threading, etc.).

The skin adhesive 1104 may include a first surface (e.g., a distal surface) configured to secure/adhere the microneedle array 1106 to the skin of the user for a period of time while the microneedle array 1106 dissolves and the active pharmaceutical ingredient (API) is delivered. In embodiments, the skin adhesive 1104 may include any suitable material configured to comfortably adhere to the skin of the patient.

The base structure 1102 provides a support for the MNA patch 1101 (e.g., the skin adhesive 1104, the microneedle array 1106, and/or optionally the identifier 1108). During application of the MNA patch 1101 to a subject, force can be applied to the base structure 1102 to cause the microneedles of the microneedle array 1106 to penetrate into tissue. Such force can be applied, e.g., via an applicator. The base structure 1102 can be flexible or rigid. For example, the base structure 1102 can be a rigid backing. The rigid backing may include a rigid material to prevent deformation of the rigid backing when the force is applied. For example, the rigid backing may resist deformation in response to the force applied to push the microneedle array 1106 into the skin (e.g., about 5 Newtons (N) to about 20 N, inclusive of subranges and values therebetween). The base structure 1102 can include a first surface on which the microneedle array 1106 is disposed and a second opposite surface to which forces can be applied to insert the microneedles into skin. The base structure 1102 may increase or ensure uniform transmission of the force to the microneedle array 1106, such that the microneedle array 1106 follows a substantially linear path into the skin during penetration. In embodiments, the base structure 1102 may have a cross-sectional area that spans a total area of the microneedle array 1106 such that force is applied uniformly to the microneedle array 1106 from the base structure 1102. For example, if the microneedle array 1106 has a first area (e.g., 15 mm by 15 mm area), the base structure 1102 may include a second area configured to cover at least the first area (e.g., at least 225 mm²). In embodiments, the base structure 1102 may include any suitable material configured to resist deformation and/or uniformly transfer force or pressure. The base structure 1102 may include, for example, a metal, a polymer, a plastic, ceramic, or a suitable combination thereof. For example, the base structure 1102 may include a polypropylene, polystyrene, nylon, polycarbonate, methacrylate, etc.

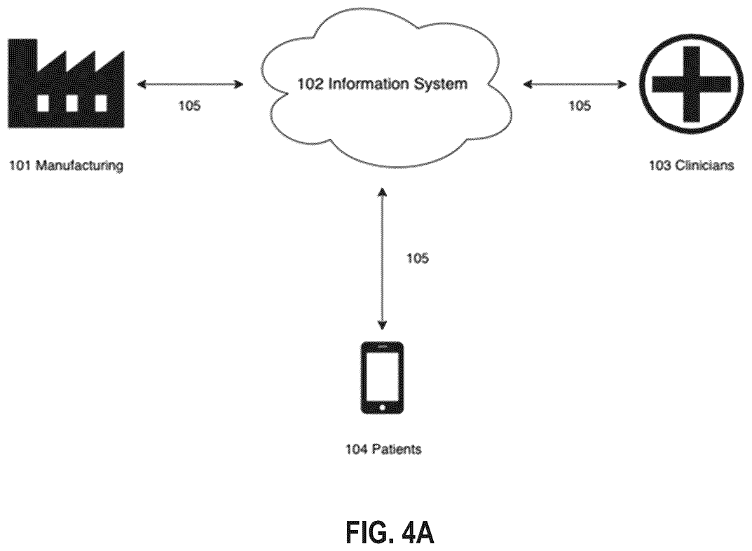

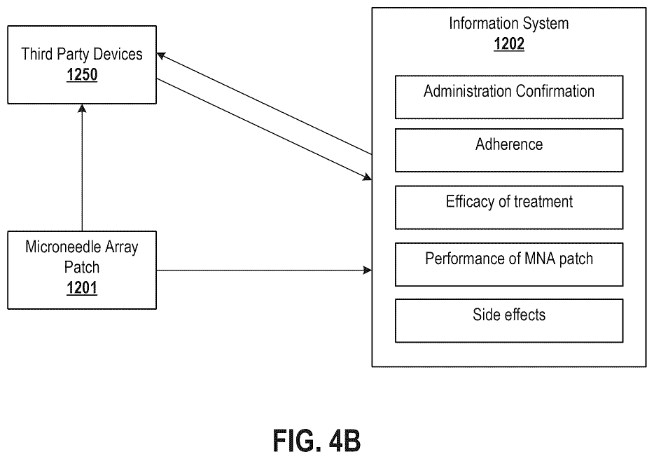

In embodiments, the base structure 1102 may include one or more components and/or materials to enable closed-loop feedback, e.g., for application, delivery, and treatment evaluation. The electronics may communicate with sensors 1112 (e.g., embedded sensors) integrated and/or adjacent to the plurality of absorbable microneedles in contact with the skin. In embodiments, the embedded sensors 1112 may detect physiological parameters including changes in skin impedance, temperature, pH, interstitial fluid levels, and/or other physiological parameters to provide confirmation to the user. In embodiments, the sensors may detect specific and/or disease specific markers (“biomarkers”) and/or concentrations of compounds to evaluate the efficacy of the absorbable MNA treatment. While the electronics can be embedded in the base structure 132, the electronics (or a portion thereof) can embed in other locations, e.g., in the microneedles (e.g., microneedle array 1106), the adhesive 1104, etc. In some embodiments, the MNA patch 1101 may include a transmitter 1114 configured to receive the signals from the sensor(s) 1112 and send the sensor data to one or more external devices. The sensor data and feedback loop for application, delivery, and treatment evaluation is described further with respect to FIG. 4B.

Optionally, the MNA patch 1101 can include the identifier 1108. The identifier 1108 can be any suitable device or component that can be used to associate a virtual identifier record, an identifier code, an identification file, or other virtual or digital identification means with the MNA patch 1101. In embodiments, the identifier 1108 can include a Radio Frequency Identification (RFID) chip, a quick-response (QR) code, a near field communication (NFC) chip, a bar code, etc. The virtual identifier record associated with a MNA patch such as the MNA patch 1101 may include an unique identifier code and associated information. One identifier record with a unique identifier code can be associated with one medical object and/or a batch of medical objects during manufacturing and/or associated directly with a patient, as described herein. In an embodiment, an identifier record with a unique identifier code is associated with a MNA patch during manufacturing of the MNA patch and the identifier record is later matched and/or paired to a patient before and/or during use of the MNA patch. In some embodiments, the identifier may be coupled to the base structure. In some embodiments, the identifier 1108 may be disposed on any portion of the medical device such as the packaging, the applicator, or any portion of the MNA patch.

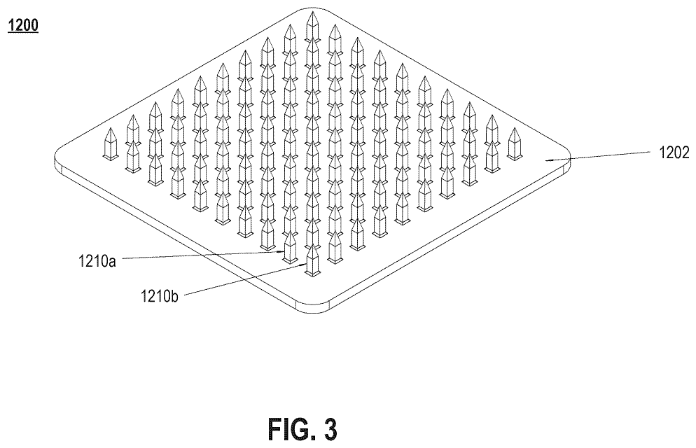

FIG. 3 depicts an example of a MNA patch 1200, according to some embodiments. The MNA patch 1200 can be structurally and/or functionally similar to other MNA patches described herein. For example, the MNA patch 1200 can include a base structure 1202 and a microneedle array including a plurality of microneedles 1210a, 1210b. The plurality of microneedles 1210a, 1210b can be the same type of microneedle or include different types of microneedles (e.g., microneedles with different lengths, sizes, configurations, structural polymers, and/or agents). The microneedle array may have any dimensions. For example, the microneedle may have a width between about 2 mm to about 30 mm, inclusive of all values and subranges therebetween. The microneedle array 140 may have a length between about 2 mm and about 30 mm, inclusive of all values and subranges therebetween.

In embodiments, the microneedles are arranged in an array that is 1 microneedle to 100 microneedles by 1 microneedle to 100 microneedles. In embodiments, the microneedles are arranged on the MNA as 10 microneedles to 50 microneedles by 10 microneedles to 50 microneedles.

In some embodiments, the MNA patch 1200 can include a first set of microneedles and a second set of microneedles different than the first set of microneedles. In some embodiments, the first set of microneedles (e.g., primary microneedles) can have a first length and the second set of microneedles (e.g., secondary microneedles) can have a second length greater than or less than the first length.

In embodiments, the microneedles can have a length of between about 50 μm and about 1000 μm, inclusive of all sub-ranges and values therebetween. In embodiments, the microneedles can have a width of between about 50 μm and about 350 μm in width, inclusive of all sub-ranges and values therebetween. In some embodiments, the first plurality of microneedles comprises microneedles 50-250 μm in length, and the second plurality of microneedles 500-800 μm in length. In embodiments, one or more of the microneedles are in the shape of a square obelisk, with a square stem structure and a pyramid tip structure. In embodiments, an agent is loaded into and/or resides in the pyramid tip structure of the microneedle of the MNA patch 1200. In embodiments, one or more of the microneedles are filleted at the base. In embodiments, the microneedles have a fillet radius of between about 10 μm and about 100 μm, inclusive of all sub-ranges and values therebetween. In embodiments, the agent is loaded into and/or resides in the stem and pyramid tip structure of the microneedle of the MNA patch 1200. In embodiments, one microneedle delivers between about 1 nanoliters (nL) and about 30 nL of agent, inclusive of all sub-ranges and values therebetween.

The microneedles can be fabricated from a structural polymer and/or an agent. In embodiments, the microneedles can be fabricated from only a structural polymer. In embodiments, the microneedles are fabricated from a structural polymer and an agent, including bioactive, non-active, and/or functional, only. In embodiments, the microneedles are fabricated from a bioactive agent only. In embodiments, the microneedles are fabricated from a non-active agent only. In embodiments, the microneedles are fabricated from a functional agent only. In embodiments, the microneedles are fabricated from an agent that is water-soluble only. In embodiments, the microneedles are fabricated from an agent that is non-water-soluble only.

In embodiments, the structural polymer is biocompatible. In embodiments, the biocompatible polymer is biodegradable or absorbable. In embodiments, the biocompatible polymer is non-biodegradable or not absorbable. In embodiments, the structural polymer is water-soluble. In embodiments, the microneedles are formed from or include biocompatible, absorbable and/or dissolvable polymers and are used for intradermal and/or transdermal delivery of various agents such as but not limited to carbohydrates, lipids, vitamins, minerals, hormones, antibodies, nucleic acids (e.g., DNA, RNA), small molecules, biologics, peptides, proteins, fillers and/or, humectants. In embodiments, the structural polymer is carboxymethylcellulose (CMC), trehalose, sucralose, polyvinylpyrrolidone (PVP), maltodextrin, silk, hyaluronic acid, polyvinyl alcohol, polyethylene glycol, poly(lactic-co-glycolic acid), poly(lactic acid), or a mixture thereof. In embodiments, the microneedles are fabricated from carboxymethylcellulose (CMC), polyvinylpyrrolidone (PVP), or hyaluronic acid (HA). In embodiments, the microneedles are fabricated from hyaluronic acid or carboxymethylcellulose (CMC). Absorbable and/or dissolvable microneedles are used for intradermal and/or transdermal delivery of various agents for applications such as, but not limited to, cosmetics, aesthetics, and/or therapeutics.

The microneedles can be designed, manufactured, and/or formulated to overcome the physical barrier of the skin, the stratum corneum. In embodiments, the microneedles are solid microneedles that create microchannels through the stratum corneum for topical application of agents to be delivered to the epidermis and dermis. In embodiments, the solid microneedles comprise a lacuna and/or channel for direct delivery of one or more agents to the skin of the subject (e.g., epidermis and/or dermis). In embodiments, the solid microneedles are coated with one or more agents that are dissolved into the epidermis and/or dermis of the skin after penetration of the stratum corneum. In embodiments, the microneedles comprise a dissolvable. absorbable, and/or biodegradable structural polymer to deliver a shelf-stable agent, either embedded or mixed in the structural polymer. In embodiments, the microneedles are non-biodegradable. In embodiments, the microneedles are fabricated from the same material as the base plate of the MNA. In embodiments, the microneedles are fabricated from different materials as compared to the base plate of the MNA. In embodiments, the microneedles are fabricated from composites, metals, plastics, water-soluble, non-water-soluble, and/or dissolvable materials.

FIG. 4A shows an example of an information system 102 that is configured to communicate with a manufacturing application 101, a patient application 104, and a clinician application 103, according to embodiments. The manufacturing application 101, patient application 104, and clinician application 103 can be implemented using one or more compute devices, including, for example, a processor (e.g., a central processing unit (CPU), a graphics processing unit (GPU), a hardware-based integrated circuit (IC) or any other suitable processing device configured to run or execute a set of instructions or codes), a memory (e.g., a random-access memory (RAM), a hard drive, or other device configured to store data, and/or code that includes instructions to cause the processor to perform one or more processes or functions), and/or one or more input/output devices (e.g., display, touchscreen, mouse, key board, button, microphone, speaker, or any other input and/or output device configured to receive. present. and/or transmit information). The information system 102 can comprise a networked application that communicates using a protocol, for example Hypertext Transfer Protocol (HTTP), a type of Transmission Control Protocol/Internet Protocol (TCP/IP), Web Sockets, or Machine-to-Machine communication (MQTT), for two-way computer networked communication. In an embodiment, the networked application communicates using HTTP 105. The information system 102 can be implemented across one or more networks by one or more compute devices, including a server, a processor, a database, etc. The system sits at the center of a star topology and is the central repository of information. The system is configured to read, write, communicate, analyze, and/or otherwise process data.

As shown in FIG. 4B, the MNA patch 1201 may include sensor(s) configured to obtain sensor data and transmitter(s) configured to transmit the sensor data to an information system 1202 (e.g., similar to the information system of FIG. 4A) for processing. The sensor data may be processed to determine at least one of administration or application confirmation, adherence or compliance, performance of the MNA patch, efficacy of treatment, or side effects. In some embodiments, the information system 1202 may be configured to process the sensor data and send information corresponding to the administration confirmation, adherence, performance of the patch. efficacy of treatment. and/or side effects to one or more third party devices 1250 such as, for example, a user device, a clinician device, a device associated with manufacturing the MNA patch, etc. In some embodiments, the processed sensor data and/or one or more notifications based on the processed sensor data can be sent to the third party devices(s) 1250. The information system may determine which third party device to send information to based on the treatment data determined from the sensor data and based on the unique identifier. For example, the information system may associate the MNA patch 1202 with a patient, clinician, manufacturer, and/or pharmaceutical company based on the unique identifier and determine to send notifications to one of these entities. In some embodiments, the information system 1202 may receive the sensor data during and/or after each administration of the MNA patch 1201.

In some embodiments, the sensor data may be processed (e.g., by the information system 1202) to confirm administration of the MNA patch 1201 has been initiated and that the MNA patch has been administered completely and correctly (e.g., “administration confirmation” or “application confirmation.”) For example, the one or more sensors may be configured to measure a change in a physiological parameter such as skin electrical impedance before and after the microneedles penetrate the skin. The sensors may include electrodes disposed on a portion of the microneedles and may obtain a baseline measurement of skin impedance when the microneedle tips are first in contact with the skin. The one or more sensors may be configured to obtain continuous (or periodic) measurements as the microneedles are pushed into the skin until a predetermined delta is measured, indicating all of the microneedles of the MNA patch have penetrated the stratum corneum and/or reached the target skin layer. In some embodiments, the one or more sensors may be configured to measure pH, temperature, transepidermal water loss in addition to or as an alternative to skin impedance. In some embodiments, a sensor may be disposed on the base structure and a sensor may be disposed on the MNA, and a position of the sensors may be tracked to determine when the base structure has been removed from the MNA.

In some embodiments, the sensor data can be processed to confirm delivery of the microneedles of the MNA patch 1201 and/or the treatment disposed therein. For example, the sensor(s) may be configured to monitor (e.g., continually or periodically) a skin condition until a predetermined delta is reached, indicating the microneedles have dissolved and that the treatment was delivered. In some embodiments, the sensor(s) may be configured to measure concentrations of one or more compounds, immediate reactive skin conditions (e.g., temperature change, pH, impedance, etc.), and/or biomarkers to determine that the microneedles have dissolved and the treatment has entered the target skin layer (e.g., stratum corneum, epidermis, dermis). Information relating to whether the microneedles have dissolved can be used to determine that the MNA patch 1201 was administered properly and the treatment has been delivered. In some embodiments, the sensor(s) can be configured to detect breakage of the microneedles, indicating the MNA patch has not been administered properly.

In some embodiments, upon determining the MNA patch has been administered (e.g., based on the sensor data,) the information system 1202 may be configured to send a notification to one or more of the third-party devices 1250. For example. the information system 1202 may send a notification to the user device such that the user device instructs the user to administer the MNA patch (e.g., if the user has not yet completed a treatment,) to remove the MNA patch and readminister a new MNA patch (e.g., if the patch was not administered properly,) and/or to contact their healthcare provider. In some embodiments, the information system 1202 may send a notification to the user device such that the user device displays instructions for properly administering a new MNA patch.

Adherence can be determined by tracking the administration of the MNA patch based on the sensor data. For example, adherence can be determined based on the application confirmation (e.g., confirmation patch was properly administered) and/or the user's prescription. In some embodiments, the information system and/or one or more of the third-party devices 1250 may be configured to store the administration confirmation information for tracking adherence and treatment progress. In some embodiments, the information system 1202 may send this information to a clinician device such that the clinician device can track treatment adherence and treatment progress. In some embodiments, the information system 1202 may send a notification to the clinician device upon determining that the user adherence is below a predefined threshold. In some embodiments, the information system may store raw and/or processed sensor data and/or information relating to administration confirmation for a particular MNA patch batch (e.g., associated with particular identifier records) and a particular patient. The administration confirmation information may be collected after administration of each MNA patch, and adherence may be determined over time as the user administered MNA patches according to their prescription.

In some embodiments, the sensor data can be used to determine an efficacy of a treatment, For example, the one or more sensors may be configured to sense physiological parameters including tissue impedance and/or disease-specific biomarkers or skin reactions after treatment delivery to determine an efficacy of the treatment. These physiological parameters can be transmitted (e.g., via the transmitter(s)) to the information system 1202 and analyzed to determine that the treatment loaded into the MNA patch is working properly. Similarly, the sensor data may be used to determine a performance of the MNA patch 1201. For example, the physiological parameters such as impedance can be used to determine at which layer under the skin the microneedles are disposed. Therefore, it can be determined whether the treatment is being delivered to the target layer of skin. In some embodiments, the sensor data may be used to detect breakage of microneedles, issues with anchoring to the skin, characteristics of the dissolution of the microneedles, and/or other issues associated with administering the MNA patch. In embodiments, the sensor may be configured to sample specific markers present in the delivery layer of the skin (e.g., by collecting and storing onboard one or more samples of the markers), and the MNA patch with the marker sample may be removed and tested outside of the skin to evaluate the efficacy of the treatment.

In some embodiments, the information system 1202 may send information associated with the treatment efficacy and/or MNA patch performance to one or more third party devices including devices associated with pharmaceutical manufacturing, MNA patch manufacturing, the clinician, the user, etc. In some embodiments, the information system may be configured to associate the treatment efficacy for particular MNA patches and batches of MNA patches based on the unique identifiers and to send this information to the third-party devices such that adjustments to manufacturing and/or formulations can be made. For example, sensor data indicating the microneedles are anchoring into an incorrect layer of tissue can be determined by the information system, and a notification can be sent to a third-party device associated with manufacturing. In some embodiments, the information system may send a notification or alert in response to a low treatment efficacy and/or administration issue being determined, e.g., to a clinician device such that the clinician can update a prescription if needed, to the user device to alert the user to issues with administering the MNA patch.

In some embodiments, the information system 1202 can use the sensor data to detect side effects from the MNA patch 140 by measuring physiological parameters indicative of swelling, scar tissue build up, immune responses, etc. For example, the sensor(s) may track scar tissue build up using impedance measures and/or immune responses based on a concentration of immune cells. Upon determining that the user is experiencing side effects, the information system can be configured to send a notification or alert to the user device to instruct the user to stop administering the treatment and/or to a clinician device informing the clinician the user is experiencing side effects.

Through the use of sensors embedded in each identifiable MNA patch 1201, the information system 1202 can provide accurate and comprehensive tracking of performance and usage of MNA patches and the agents therein.

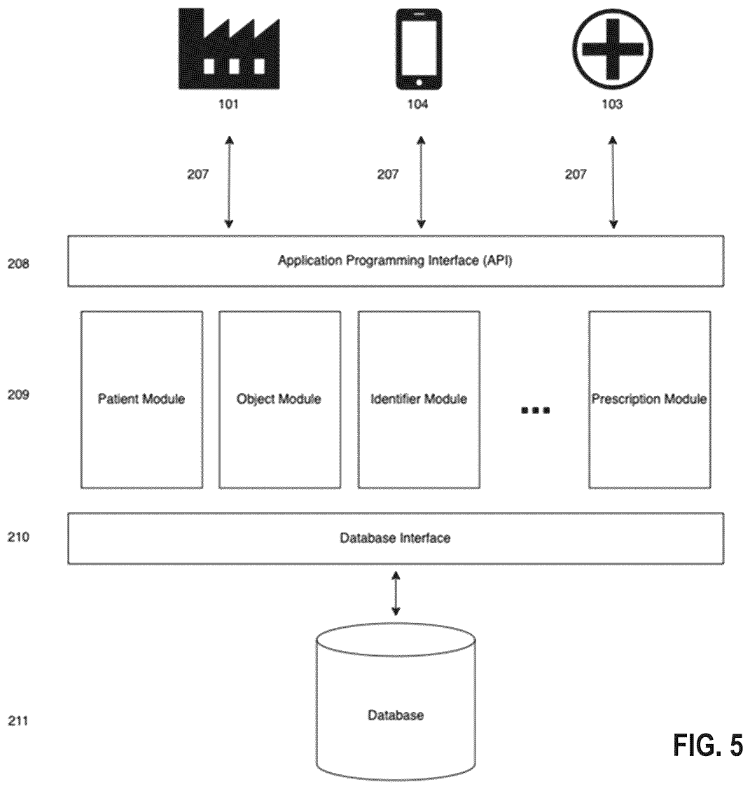

As seen in FIG. 5, an information system (e.g., information system 102) can have multiple software layers, according to embodiments. In some embodiments, the information system can have a top layer 208 that implements an Application Programming Interface (API). The top layer 208 (or API layer) can execute instructions that implements a server process responsible for handling, managing, and/or authenticating HTTP network traffic 207, e.g., by sending and receiving authorized data, and/or rejecting unauthorized access (e.g., unauthorized API requests). The API can include several different endpoints, each responsible for processing and forwarding certain types of requests and data. In one embodiment, the API is the exposed boundary of the information system, managing and directing data as it passes from the manufacturing application 101, patient application 104, and clinician application 103 to the appropriate module (e.g., software module, process, and/or function being implemented by hardware) in the business logic module layer 209.

The business logic module layer 209 of the information system sits below the API layer. The business logic module layer contains one or more modules, each responsible for processing a specific type of data and request. A module can be a software module, process, and/or function being implemented by hardware (e.g., a processor or server). Each module comprises various software subroutines, logically grouped based on the module type. Each module is the intermediary between API requests from the API layer 208 and retrieving or writing data/information to a database (e.g., persistent storage) 211 of the information system. The data/information can be sent to and/or retrieved from the database 211 using a database interface 210. In one embodiment of the system, the business layer includes modules for processing patient information, object information (e.g., information of one or more microneedle array patches), identifier information (e.g., identifier records of one or more microneedle array patches), shipping information (e.g., shipping information of one or more microneedle array patches), prescription handling (e.g., prescription data of one or more microneedle array patches), recall handling (e.g., recall data of one or more microneedle array patches), logging, and/or user authentication.

The database interface layer 210 controls communication between the business logic modules and the database. Routing database operations through the interface ensures data consistency, improving system modularity and security. For example, the database interface prevents two modules from changing the same data simultaneously. The database interface can allow reading of data and/or writing of data in database 211, and can organize requests based on preset criteria. The database interface can ensure that each module can read and write to the database 211 so that no business layer module monopolizes the database 211. The database interface also provides accountability by logging operations sent to the database, e.g., by logging the specific module that requested the data and/or sent the data, by logging when the request was sent, by logging when data in the database 211 is changed, by logging the reasons for the change in the data in the database 211.

The last layer in the information system is the database 211, for a device configured to provide persistent patient and/or object data storage. The database can store the data and/or ensure that data is available and/or correct.

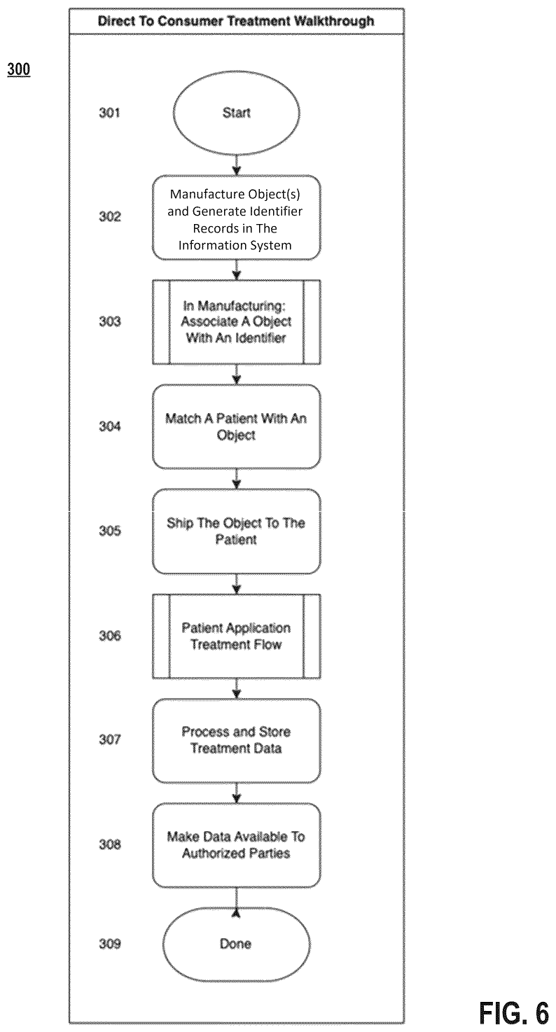

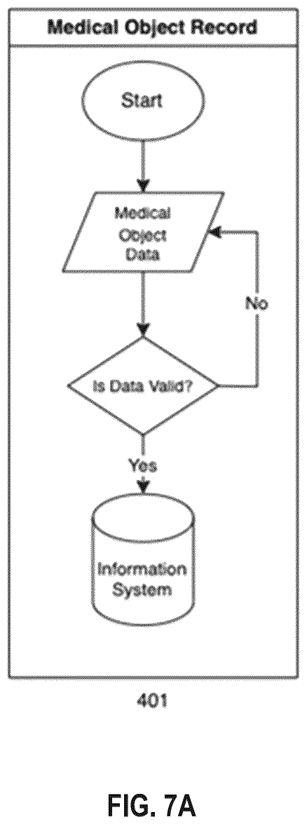

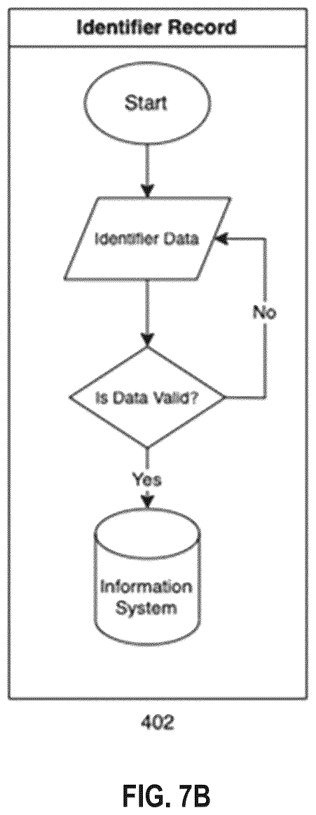

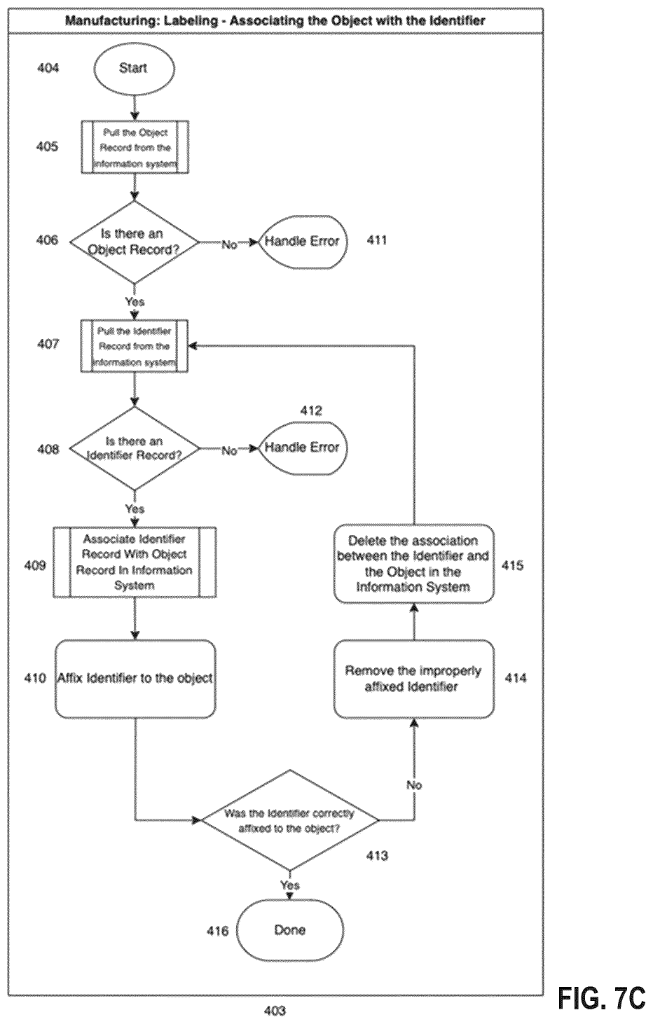

FIG. 6 shows a flow chart of a method 300 for tracking treatments and/or objects from manufacturing to use, according to embodiments. In some embodiments, one or more portions of the flow depicted in FIG. 6 can be implemented using an information system as described herein (e.g., information system 102) and/or one or more compute devices running and/or implementing one or more of a manufacturing application (e.g., a manufacturing application 101). a clinician application (e.g., clinician application 103), and/or a patient application (e.g., patient application 104). At 301, a manufacturer can be ready to make the medical object and/or treatment and has the components and materials needed. At 302 (and described in more detail in FIGS. 7A-7B at 401 and 402), the method 300 includes manufacturing a medical object and generating and adding an identifier record to the information system. For example, a medical object may be a drug delivery device (e.g., a microneedle array patch). In some embodiments, a unique identifier such as, for example, a Universally Unique Identifier (UUID) may be generated during manufacturing (e.g., after the identifier such as an RFID chip is added to the microneedle array patch.) The identifier record can include an unique identifier code and associated information. One identifier record with an unique identifier code can be associated with one medical object and/or a batch of medical objects during manufacturing (e.g., at 302) and/or associated directly with a patient (e.g., at 304 described below). In an embodiment, an identifier record with an unique identifier code is associated with one medical object during manufacturing (at 302) and the identifier record is later matched and/or paired to a patient (e.g., associated with a patient record) before and/or during use of the medical object (at 304). In some embodiments, the unique identifier can be read (e.g., by a processor of one or more devices associated with manufacturing and/or by the information system) to confirm the unique identifier was properly written.

At 303, the method 300 includes associating (e.g., at the information system) the medical object with the identifier record. In some embodiments, the method 300 can include affixing an identifier to the object. In some embodiments, the information system can receive the unique identifier configured to be fixed to the object from a device associated with manufacturing the object. In some embodiments, the information system can receive the identifier during or after manufacturing of the object. In some embodiments, the identifier (e.g., identifier 1108) can be affixed or attached to the medical object. The identifier can be a Radio Frequency Identification (RFID) chip, a quick-response (QR) code, a bar code, a near field communication (NFC) chip, etc. The medical object with the identifier is then associated with an identifier record. This step is further described with respect to FIGS. 7A-7C. In some embodiments, once it is confirmed the unique identifier was properly written, the unique identifier may be associated with a current batch number in the information system.

At 304, the method 300 includes matching a patient with the medical object. For example, a patient may submit their prescription for a prescribed treatment, e.g., at a store, pharmacy, or other location, and information regarding the patient and their prescription can be sent to the information system. The information system can then pair or associate the information regarding the patient and their prescription to a specific medical object for the prescribed treatment. In some embodiments, the information system can associate the patient with the object (e.g., a MNA patch) based on a prescription received from a device associated with a clinician or prescribing entity (e.g., the clinician device or clinician application).

At 305, the method 300 includes shipping the medical object to the patient, after the medical object has been associated with the information regarding the patient and their prescription. In some embodiments, the shipment may occur through traditional pharmaceutical distribution channels or generally available commercial shipping methods.

At 306, the method 300 includes the patient using the treatment. The patient can have an application (e.g., patient application 104) implemented on a compute device, such as a personal digital device (e.g., a computer, smartphone, smartwatch, tablet computer), to facilitate treatment using the medical object. In one embodiment, the patient application can capture physiological information regarding the patient (e.g., the patient's vitals) before, during, and/or after use of the medical object. FIG. 7 provides a more detailed view of the patient software treatment flow. The patient application can also capture usage of the medical object by the patient. For example, the patient application can record when a patient has used a microneedle array patch. In some embodiments where the microneedle array patch may include a sensor as described herein, the patient application can also receive information from the sensor to determine that the microneedle array patch has been unpackaged, has been applied to skin, has been used according to prescribed requirements (e.g., applied to the right region of tissue, applied for the right duration of time, etc.), has been removed from skin, has been discarded, and/or the like. For example, the microneedle array patch can have a sensor that can detect when the microneedle array patch has been applied to a skin surface (e.g., via measuring temperature, impedance, skin conductance, etc.), and for how long it has been applied. In some embodiments, the user device can scan and/or send the unique identifier to the information system prior to administration of the microneedle array patch such that the information system can confirm the MNA patch is valid and the correct MNA patch.

At 307, the information system can store patient treatment data including, for example, the information regarding the physiological data of the patient and the usage information or data of the medical object captured at 306. This data (e.g., the unique identifier, the prescription, the usage data, etc.) can be sent from the patient application to the information system—for example, to an API endpoint (e.g., at API layer 208) associated with a patient module (e.g., a software module that receives and/or processes information regarding a patient)—using an HTTP request. In some embodiments, the API can route the treatment data to one or more modules (described in FIG. 5) configured to process the treatment data. For example, from the API endpoint. the data can be routed to the patient module, where it can be processed and sent to the database for storage. In an embodiment, the patient application can send the patient information directly to the API endpoint.

At 308, the information system can provide the patient treatment data to one or more authorized parties. In some embodiments, the information system can be configured to send patient treatment data to one or more compute devices that manage patient health records, such that the patient treatment data can be added to the electronic health/medical record (EMR) of the patient. In some embodiments, the information system can send adverse reaction data of a patient to clinicians, manufacturers, and/or regulators. In some embodiments, the information system may retrieve treatment data from the database and send the treatment data to the one or more authorized parties (e.g., a clinician device or a user device.) The information system, by automatically making this information available to one or more other systems, can function as a centralized system that receives patient treatment data and uses it to update other decentralized record systems (e.g., electronic record management systems). The information system can also receive information regarding recalls, safety issues, updated treatments, etc., and send this information to patients, clinicians, or other interested parties to facilitate usage of and/or exchange of information regarding the medical object.

In some embodiments, one or more devices (e.g., the user device or the clinician device) may be configured to monitor treatment progress based on treatment data stored in the database. For example, treatment progress may be monitored based on usage data and/or patient information.

While FIG. 6 depicts several steps involved in the process of tracking treatments and/or objects from manufacturing to use, it can be appreciated that not all of the steps need to be implemented, and that in some embodiments, one or more steps may be set in a different order. Moreover, it can be appreciated that information systems as described herein (e.g., information system 102) can be configured to perform software steps associated with receiving, processing, associating, and/or distributing information (e.g., portions of steps 302. 303, 304, 306, 307, 308), and that other steps (or portions thereof) can be performed by a user or operator. Variations in order of steps and/or inclusion or exclusion of one or more steps are contemplated within the scope of the present disclosure.

FIGS. 7A-7C depict the process flow of associating an object with an unique identifier, according to embodiments. In some embodiments, the process flow depicted in FIGS. 7A-7C can be implemented using one or more compute devices, including information systems as described herein (e.g., information system 102) and/or a compute device running a manufacturing application (e.g., manufacturing application 101).

FIGS. 7A-7B, at 401-402, show steps associated with generating and adding object and identifier records to the information system. At 401, the medical object data can be generated and validated, and then stored to the information system (e.g., database 211 of the information system). At 402, an identifier record including an unique identifier can be generated and validated, and then stored to the information system (e.g., database 211). When these are generated, certain protocols can be used to check whether the medical object data and/or identifier record is valid (e.g., does it comply with certain criteria, is it non-duplicative of other records, etc.).

FIG. 7C then depicts the process of adding the identifier to the object (e.g., affixing the identifier to a microneedle array patch) and associating the identifier and the identifier record associated with the identifier with the object. At 404, an identifier is ready to be affixed to an object, and an object is ready for labeling. At 405, the manufacturing application may pull the object record from the information system (e.g., by sending an API request to the information system). The manufacturing application then may confirm. at 406. that the object has an object record and is ready to be labeled. If there is a problem with the object record. the handle error subroutine is called, at 411. which may prompt the process to retry an attempt to register the object and/or notify the information system of a failure. Further, an identifier may be selected to pair with the medical object. The manufacturing application, at 407, may pull the identifier record from the information system.

At 409, the information system, the identifier record. and the object record are associated with each other. An object may be associated with a singular and/or multiple identifiers and an identifier may be associated with a singular and/or multiple objects. In one embodiment, an object is associated with one identifier and the identifier is associated with the one object, in a one-to-one relationship to create a singular object unique identifier record. For example, an object can be associated with only one identifier and that identifier can be associated with only that object. Alternatively, multiple objects manufactured as a batch can be associated with a single identifier to create a singular batch unique identifier record.

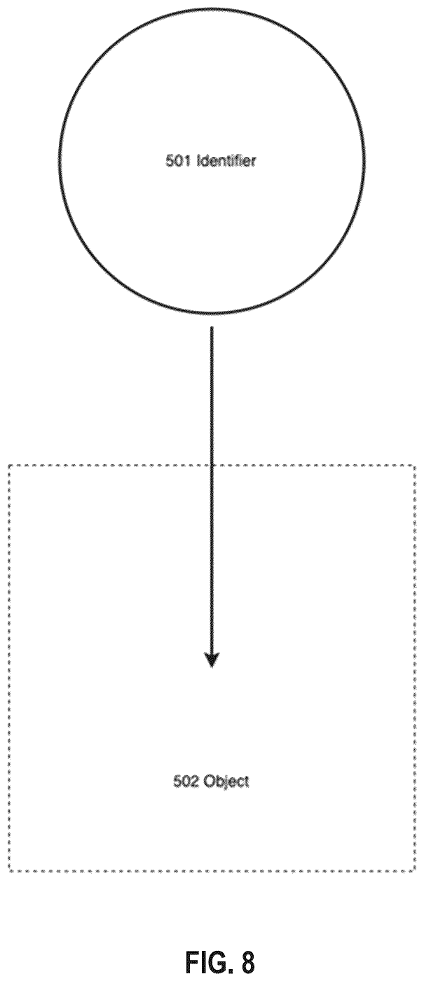

The identifier may then be affixed to the object, at 410. The identifier may be affixed to the object with one or more of adhesive (e.g., ultraviolet cured, contact, cyanoacrylate, polyvinyl acetate, and/or polyurethane), geometric fits (e.g., snap and/or interference), over molding, solder, Velcro or hook-and-loop fastener, magnets, clips and/or clamps. FIG. 8 schematically depicts an identifier 501 being affixed to an object 502. During inspection after the identifier is fixed to the object, if the identifier is affixed incorrectly. there is a process for removing the identifier, at 414. Such a process can include removing the object identifier association (e.g., the association between the object record and the identifier record) from the information system, at 415, and returning to the step of 407 to pick a new identifier. If the identifier was affixed correctly, the process of adding a label to the object is now done, at 416. As described above, the object can include a medical object such as a microneedle array patch, and the identifier can include a RFID chip or other type of physical device that can be used to identify the object. The identifier may be affixed to the microneedle array patch during manufacturing, and the unique identifier code can correspond to a specific microneedle array type and/or treatment (e.g., specific size or configuration of a microneedle array patch, specific agent or agents being delivered via the patch, etc.).

While FIGS. 7A-7C depict several steps involved in the process of associating an object with a unique identifier, it can be appreciated that not all of the steps need to be implemented, and that in some embodiments. one or more steps may be set in a different order. Moreover, it can be appreciated that compute devices as described herein (e.g., information system, or a compute device running a manufacturing application 101) can be configured to perform software steps associated with receiving, generating, associating, and/or processing information (e.g., portions of steps depicted in FIGS. 7A-7C), and that other steps (or portions thereof) can be performed by a user or operator. Variations in order of steps and/or inclusion or exclusion of one or more steps are contemplated within the scope of the present disclosure.

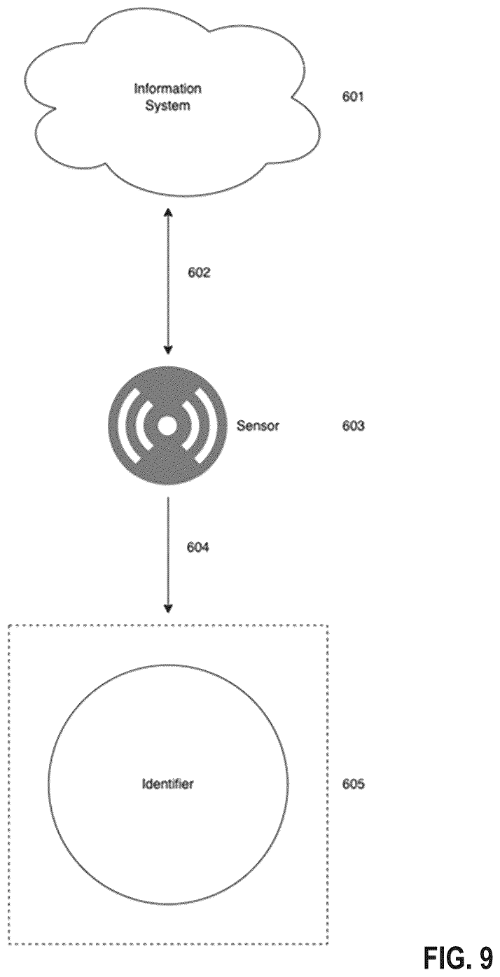

FIG. 9 depicts a sensor 603 that is used in a manufacturing process, according to embodiments. The sensor 603 is configured to read, at 604, an identifier 605 in preparation for pairing the identifier with an object (e.g., a medical object such as a microneedle array). The identifier can be similar to other identifiers described herein, including, for example, identifier 1108. In one embodiment, the identifier 603 may comprise an RFID chip in which a unique identifier code has been written and/or assigned to the identifier during preparation for pairing with an object. In another embodiment, the identifier 605 may comprise a barcode or QR code in which a unique identifier code has been randomly generated and printed on the identifier label. In another embodiment, the identifier 603 can include a near field communication (NFC) chip and NFC protocols can be used for reading the unique identifier code in the NFC chip. In one embodiment, the sensor 603 may be located on a device and/or equipment that is responsible for or associated with implementing a manufacturing application (e.g., manufacturing application 101). In another embodiment, the sensor 603 may be a part of a separate device that is configured to read the unique identifier code of the identifier. This separate sensor 603 may read the identifier 605, e.g., via RFID communication protocols and/or scan the unique identifier code printed on the identifier label (e.g., QR code or barcode), and then send this information to a compute device running the manufacturing application and/or an information system 601 (e.g., structurally and/or functionally similar to other information systems described herein). In some embodiments, the manufacturing application (or separate device including the sensor), via two-way communication at 602, the information system 601 to retrieve the identifier record for the identifier 605.

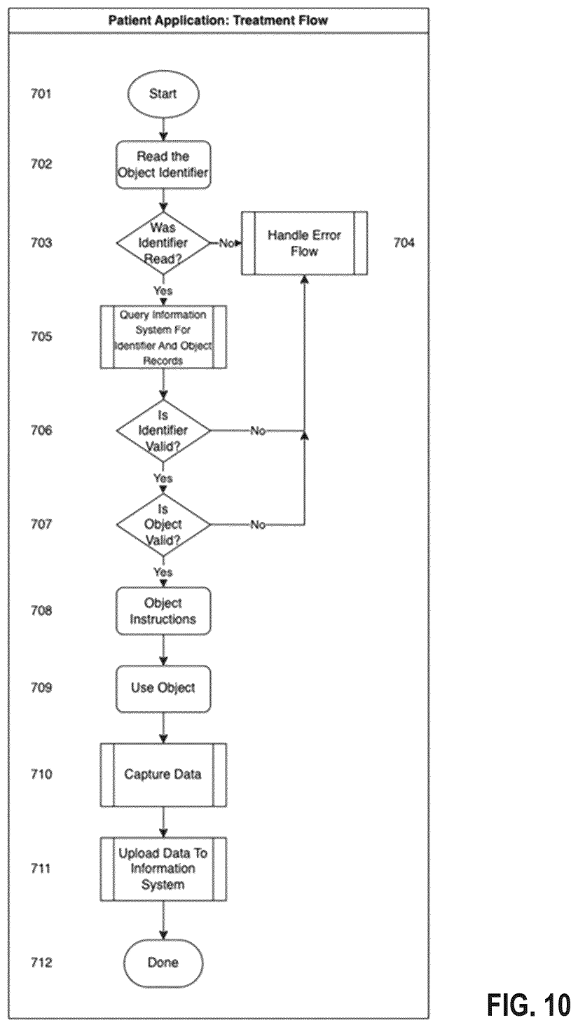

FIG. 10 shows a software treatment flow for a patient application, according to embodiments. In some embodiments, the process flow depicted in FIG. 10 can be implemented using one or more compute devices, including a compute device running a patient application (e.g., patient application 104). While the process flow is described with reference to a microneedle array patch, it can be appreciated that the process flow can be applicable to other medical objects, including medical objects that are not microneedle array patches.

At 701, the patient with a medical object (e.g., microneedle array patch) can access the patient application (e.g., patient application 104). In some embodiments, the patient application may be used and/or run using a compute device such as a personal digital device or user device (e.g., a computer, mobile device, smartphone, smartwatch, tablet computer). For example, the patient application can be run on a smartphone. The mobile device can be configured to communicate with an information system (e.g., information system 102), where the patient can be registered and have a patient record stored.

At 702, the patient may use the patient application to activate a sensor (e.g., a RFID reader, NFC reader, barcode scanner, camera, and/or other device configured to detect and read an identifier) to read an identifier (e.g., identifier 605, 1108, or any other identifiers described herein) of the medical object. In some embodiments, the sensor can be integrated with and/or disposed on a compute device running the patient application, such as a mobile device, while in other embodiments, the sensor can be a separate device configured to read the identifier affixed to the object.

If, at 703, the sensor cannot read the identifier, a subroutine that implements a handle error flow is executed, at 704. The subroutine may include directing (e.g., through the patient application) the patient to customer support and/or troubleshoot information. Some example reasons for the identifier not being read can include the patient application lacking sensor permissions from the operating system of the mobile device, the mobile device not having the necessary sensor to read the identifier, and/or the identifier being damaged.

In one embodiment, after the sensor reads the identifier, the patient application, at 705, can request the identifier and object records from the information system using an HTTP GET request to an identifier API endpoint (e.g., in layer 208 of FIG. 5). The information system may then process the request (e.g., via the identifier module at layer 209) and send a response. That response may be the record for the identifier and the object, or an error, such as an identifier validity error, a recall issue, safety issue, or other issue associated with the identifier and/or object.

The patient application, at 706 and 707, checks if the response from the information system indicates that the treatment can proceed and/or there is an error. The patient application may include a query (e.g., a real-time or near real-time query) to confirm the medical object (e.g., MNA patch) is valid and that the medical object is the correct device (e.g., associated with patient or user, not recalled or expired, not identified as a counterfeit, etc.). In some embodiments, the user device may be configured to receive the unique identifier of a MNA patch from a sensor configured to read the unique identifier. The patient application may be configured to instruct the user to scan the unique identifier of the MNA patch. The patient application may then be configured to read the unique identifier and initialize the query with the information system. For example, the processor of the user device may be configured to query the information system to confirm the identifier record and the microneedle array patch associated with the unique identifier are valid. The patient application may send the information system the unique identifier of the MNA patch and a patient identifier, e.g., using a API request. In some embodiments. the information system may confirm the unique identifier is valid, e.g., the MNA patch is not counterfeit, expired, or revoked for safety reasons. In some embodiments, the compute device running the patient application is configured to query the information system to confirm the identifier and the microneedle array patch associated with the identifier are valid by sending the information system the identifier and information of the user such that the information system, in response to receiving the identifier and the information of the user, retrieves the identifier record associated with the identifier and a patient record associated with the user and determines that the identifier record and the patient record are associated with one another.

If there is a validity error with the object and/or identifier, the handle error flow subroutine at 704 is called. For example, a counterfeit object is one reason for an invalid response from the system. In one embodiment, the handle flow subroutine may notify the patient and/or user that the object is counterfeit and direct the patient to dispose of the counterfeit object. The patient application then may register the instance of the counterfeit object and request the patient to order an additional object that is registered in the information system. Alternatively, or additionally, if an object is recalled or has passed its expiration date, the information system can report the object as invalid.

In one embodiment. if the identifier and object are valid, the patient application presents the object instructions to the user (e.g., to administer the MNA patch), at 708. For example, the information system, after determining that the identifier and object are valid, can send a response to the compute device running the patient application that indicates to the patient application that the identifier and the object are valid. The compute device (or processor thereof) running the patient application can then present the instructions to the user to administer the medical object and, in embodiments, a MNA patch. In some embodiments, the instructions that are presented to a user may differ based on the type of microneedle array patch. For example, multiple types of microneedle array patches may be available (e.g., different ones including different configurations of microneedles, different size of needles, and/or different agents loaded thereon), and the instruments for use for these different types of microneedle array patches may differ. Therefore, after receiving a response from the information system indicating that the identifier and microneedle array are valid. the patient application may determine which instructions to present to the user based on the type of microneedle array patch. In one embodiment, wearable sensors can start capturing patient vitals (e.g., physiological information of the patient, such as heart rate, photoplethysmogram (PPG), temperature, SpO₂), which can be used to set a baseline for the patient's vitals before treatment. In some embodiments, the wearable sensors can be operatively coupled to the compute device running the patient application, such that the patient vital data captured by the wearable sensors can be transmitted. via the patient application. to the information system (e.g., the API as described with reference to FIG. 5). After the patient receives the treatment instructions, they can use the object (e.g., administering a treatment or taking a drug), at 709.

In embodiments, at 710, the treatment data and associated metadata (e.g., time of use, duration of use, patient information, vitals, the unique identifier, etc.) can be captured from sensors and sent to the patient application. For instance, if the patient is using wearable sensors, in one embodiment, the patient application may capture the patient's heart rate, blood pressure, skin conductance, temperature, and/or any embedded electronic data from the identifier itself. Alternatively, or additionally, embedded electronics or sensors in the identifier and/or on the microneedle array patch may measure biomarkers or vitals in the patient's interstitial fluid of the skin. Still alternatively, or additionally, the patient application may allow for direct data capture for the patient to submit additional feedback exclusive of wearable sensor data, including but not limited to symptoms, feedback and/or reactions, additional benefits outside the predetermined use case, patient adherence, patient comfort, ease of use, and/or other feedback.

In this example embodiment, after the treatment finishes, the patient application may gather the treatment data (e.g., patient vitals, usage information, etc.) and, at 711, upload the treatment data to the information system for processing and storage. The patient application treatment flow can then end at 712. In some embodiments, patient application may be configured to track or monitor treatment progress and/or medication use based on treatment data. The client software application may be configured to track when the user takes, misses, or skips medication (i.e., compliance information.) In some embodiments, the patient application may be configured to record, in response to receiving the unique identifier of the MNA patch, the MNA patch has been administered and calculate a compliance score of the user based on a prescription of the user and usage data (e.g., recorded administrations) of one or more MNA patches associated with the user. In some embodiments, compliance information may be displayed in a calendar view. In some embodiments, the processor of the user device may be configured to calculate a compliance score. The compliance score may be non-linear and weighted by a severity measure indicating a severity of missing a dose of a particular medication

While FIG. 10 depicts several steps involved in the process of patient treatment flow, it can be appreciated that not all of the steps need to be implemented, and that in some embodiments, one or more steps may be set in a different order. Moreover, it can be appreciated that compute devices as described herein (e.g., information system 102 and/or a compute device implementing patient application 104) can be configured to perform software steps associated with receiving, analyzing, validating, capturing, and/or sending information (e.g., portions of steps depicted in FIG. 10), and that other steps (or portions thereof) can be performed by a user or operator. Variations in order of steps and/or inclusion or exclusion of one or more steps are contemplated within the scope of the present disclosure.

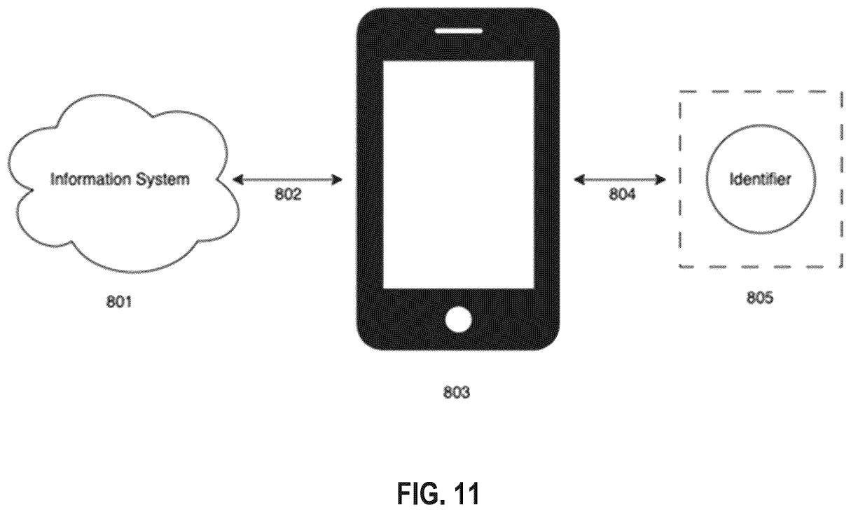

FIG. 11 shows an example of how the patient application device 803 (e.g., compute device running a patient application) may use its sensors to communicate via a communication channel 804 with an identifier 805 (e.g., structurally and/or functionally similar to other identifiers described herein) and via a communication channel 802 and/or network with the information system 801 (e.g., structurally and/or functionally similar to other information systems described herein).

In one embodiment, the identifier 805 is readable by digital sensors. A unique short identifier, such as a Universally Unique Identifier (UUID) v4, can be used to reference the object record in the information system and encoded on a QR Code, RFID, or NFC chip. In some embodiments, a human readable shorter code, such as a 16-digit alphanumeric code, can be printed on the device as a human readable backup to the digitally encoded identifier.

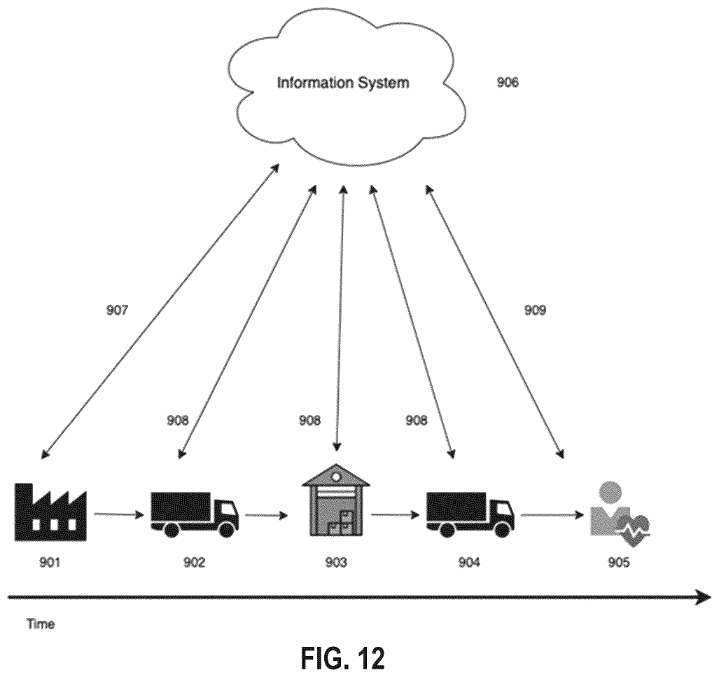

FIG. 12 illustrates one embodiment of the system for tracking an individual medical object over time, e.g., from manufacturing to use. This embodiment shows an example of the complete tracking of the medical object's lifecycle. In one example, a patient is sent a one-time-use drug treatment or product. This treatment, product, and/or object can be a microneedle array patch. The microneedle array patch may comprise a biodissolvable and/or biodegradable needle material, e.g., to avoid complex sharp disposal. The biodissolvable and/or biodegradable needle material may allow for an agent (e.g., drug, treatments, biologics, and/or other bioactive or inactive agents) to be embedded in solid-state and embedded in the needle structural material. Solid-state embedding may allow for these microneedle array treatments to be stored at room temperature (e.g., be shelf-stable) and remove the current necessity of cold chain storage for liquid drugs, treatments, and/or biologics.

In embodiments, during manufacturing 901, identifier and medical object information is added to an information system 906 (e.g., structurally and/or functionally similar to any of the information systems described herein, including information system 102) by sending the data to the information system's identifier and object API endpoints 907. In some embodiments, the data is sent as a structured JavaScript Object Notation (JSON) object. The medical object information may include, for example, object type, manufacturing lot, instructions, disclosures, safety data, expiration date, regulatory information, and/or traceability data. Once the object passes quality assurance and is released for use, it can be shipped 902, 903, 904 (e.g., to a pharmacy). During transit, the information system may be updated with the object's location, e.g., by generating and sending a location record 908 to the information system. Any stationary location between manufacturing and the patient (e.g., a shipping facility, warehouse, pharmacy, or retail store) may trigger that a location record 908 be sent to the information system. When the patient uses the medical object at 905, the information system may be queried with the object identifier for the object information. Before, during, and after treatment, the patient's vitals may be recorded and/or sent to the information system for processing and storage, at 909.

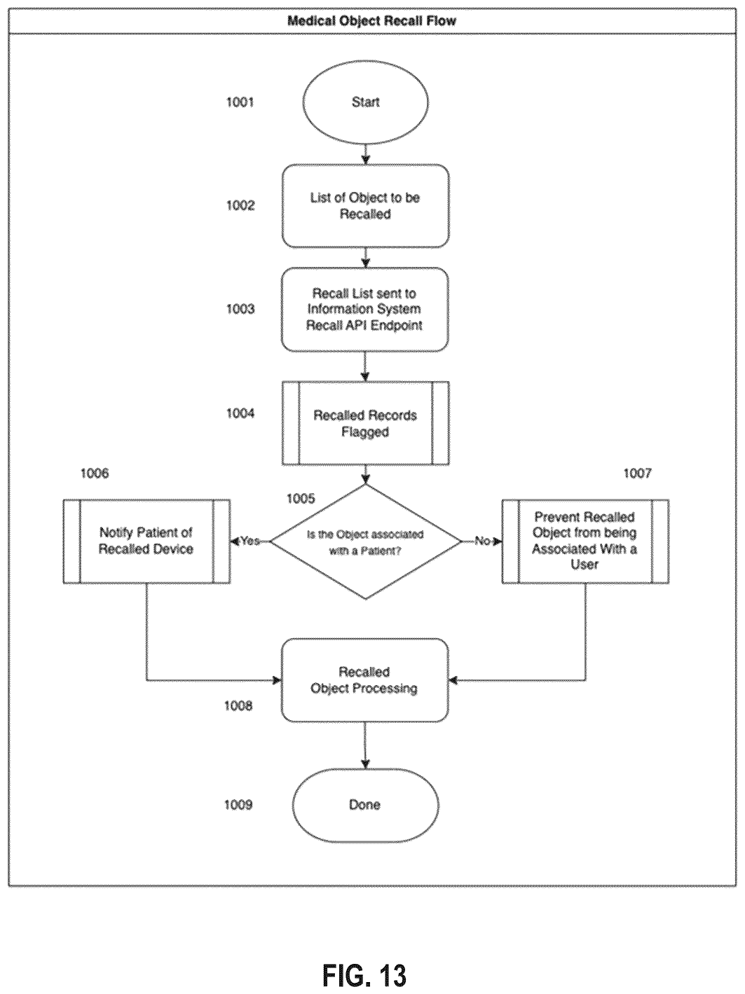

In one embodiment. the information system can issue real-time medical object recalls. A recall module in the business layer of the information system (e.g., layer 209 in FIG. 5) can store the recall subroutines. As seen in FIG. 13, at 1001, the manufacturer may determine to issue a recall. The manufacturer and/or drug developer, at 1002, compiles a list of objects to recall. At 1003, the recall list is sent as structured JSON data to the information system's recall API endpoint (e.g., layer 208 in FIG. 5). While this specific example is provided here, it can be appreciated that other suitable data types can be used to transmit the recall list. From there, the API endpoint passes the recall JSON data to the recall subroutine in the recall module, located in the business layer of the information system (e.g., layer 209 in FIG. 5). The recall subroutine is responsible for flagging (e.g., marking as recalled) the recalled object records in the information system, at 1004. The recall subroutine then checks to see if the object records are associated with a patient, at 1005. If yes, then another subroutine is triggered, at 1006, to notify the patient of the object recall. In the case where an object is not yet associated with a user in the information system, the system removes the object from the patient matching subroutine, at 1007, preventing the recalled object from being associated with a user. Then, the recalled object is processed, at 1008. It may need to be retrieved, returned, and disposed of, The process ends at 1009.

In one embodiment of the information system (e.g., information system as described with respect to any of the figures above), a patient's adverse event can trigger the patient software to generate an event report. That report can be sent as structured JSON data to the information system's adverse event API endpoint. From there, the API can pass the structured data to the adverse event module in the business layer of the information system (e.g., layer 209 in FIG. 5). There, a subroutine can be called to send a notification of the event to the patient's Electronic Health Record and/or the drug manufacturer. In one embodiment of the information system, the adverse event module can have a subroutine for sending reports to the Food and Drug Administration (FDA) Adverse Event Reporting System (FEARS) or other reporting system.

In one system embodiment, patient treatment data inclusive of patient vitals and/or direct patient feedback as described herein can be used to support post-marketing and/or post-approval surveillance studies. By capturing patient vitals before, during, and after use, as well as metadata related to the treatment, a rich dataset can be available for monitoring treatment effects. In some embodiments, a data analysis module (e.g., in layer 209 of an information system) runs statistical analysis and machine learning on historical treatment data. In one embodiment, the treatment data may be analyzed to identify patient deviations from the prescribed dosing protocols and/or assess potential additional benefits. This analysis can inform adjustments to the currently acceptable or optimal dosing intervals, e.g., especially considering these intervals might change or become more precise with a larger sample size than the initial clinical trial. The insights gained can support, for example, updating product labeling with new safety information, issuing safety warnings, and changing usage guidelines. leading to better patient health outcomes. In some embodiments, the information system can send the information to a physician, and a physician can adjust the prescription of a user based on this information.

Another application of the data analysis module (e.g., in layer 209 of an information system) can explore new treatment indications for a treatment based on the additional patient feedback captured at 710. By having a large dataset of treatment usage data and metadata, studies can examine whether expanding the medical conditions treated by the intervention is warranted.

In one embodiment of the information system, there can be a prescription module (e.g., in layer 209 of an information system) with a matching subroutine for matching patients to medical objects using electronic prescriptions (e.g., ePrescription). The patient can be issued a prescription by their healthcare provider. The provider can enter the prescription details, including patient and treatment information. The information system has an API endpoint for receiving prescriptions securely. Upon receiving the prescription, the information system matches the patient with a medical object. In another embodiment, a new prescription may trigger a Just In Time (JIT) manufacturing process. Then, the object is packed and shipped to the patient.

In another embodiment, the information system can have a secure API endpoint for third parties, such as pharmacies and hospitals, to order treatments. The treatments are shipped wholesale for later distribution and association to patients via prescriptions.

In one embodiment of the information system, it can integrate with other information systems, such as Electronic Health Record (EHR) systems. This integration is implemented in a business layer module that communicates with third-party EHR systems through their API and/or software development kit (SDK). The integration can allow the information system to update a patient's health record with treatment data and metadata, such as patient vitals, usage data, and/or other feedback as described herein, with each treatment use. For example, when a patient takes a treatment at home, such as a drug injection, the information system can update the patient's electronic health record.

One example use of this treatment data is to provide the patient's medical team with a way to monitor the patient's compliance with the treatment protocol. This treatment data can allow clinicians to provide patient intervention, such as, for example, corrective action and patient education, to correct any treatment errors, leading to better patient outcomes. For example, if a patient is supposed to take a treatment every seven days, but instead, they take the treatment at irregular intervals, their healthcare team can notify them of the correct treatment protocol.

While specific examples have been provided in the figure for example and illustrative purposes, it should be clear that variants can be constructed without limitation and based on the teachings herein.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer. in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either.” “one of,” “only one of,” or “exactly one of.” “Consisting essentially of,” when used in the embodiments. shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. A system, comprising:
a microneedle array patch including:
a base structure including first and second flat surfaces;
a plurality of microneedles disposed on the first flat surface of the base structure, the plurality of microneedles configured to extend out from the first flat surface, the base structure configured to support the plurality of microneedles when force is applied against the second flat surface to insert the plurality of microneedles into skin;
a first sensor disposed on a microneedle of the plurality of microneedles, the first sensor configured to measure one or more physiological parameters when the microneedle array patch is inserted into the skin of the user;
a transmitter disposed on the microneedle array patch and configured to send the measurements of the one or more physiological parameters to an information system; and
an identifier disposed on the microneedle array patch;
a second sensor separate from the microneedle array patch and configured to read the identifier; and
a processor operatively coupled to the second sensor, the processor configured to:
receive, from the second sensor, the identifier of the microneedle array patch, wherein the identifier is associated with an identifier record;
query the information system to confirm the identifier and the microneedle array patch associated with the identifier are valid;
in response to receiving a response from the information system indicating that the identifier and the microneedle array patch are valid, present instructions to the user to administer the microneedle array patch; and
receive, after the user administers the microneedle array patch, treatment data related to the one or more physiological parameters from the information system.

2. The system of claim 1, wherein the plurality of microneedles includes first and second sets of microneedles, the first set of microneedles having a first length and the second set of microneedles having a second length greater than the first length.

3. The system of claim 2, wherein the first length is in a range of between about 500 and about 800 μm and the second length is in a range of between about 50 μm and 250 μm.

4. The system of claim 1, wherein the processor is configured to query the information system to confirm the identifier and the microneedle array patch associated with the identifier are valid by sending the information system the identifier and information of the user such that the information system, in response to receiving the identifier and the information of the user, retrieves the identifier record associated with the identifier and a patient record associated with the user and determines that the identifier record and the patient record are associated with one another.

5. The system of claim 1, wherein the identifier is at least one of a quick-response (QR) code, a bar code, a radiofrequency identification (RFID) chip, or a serial number.

6. The system of claim 10, wherein in the information system is configured to:
process the measurements of the one or more physiological parameters to obtain the treatment data, the treatment data including information associated with at least one of:
confirmation of administration, efficacy of treatment, performance of the microneedle array patch, or side effects.

7. The system of claim 6, wherein the one or more physiological parameters include skin impedance, pH, a level of interstitial fluid, or a concentration of biomarkers.

8. The system of claim 1, wherein the processor is further configured to:
record that the microneedle array patch and one or more additional microneedle array patches have been administered by the user based on the treatment data received from the information system; and
calculate a compliance score of the user based on a prescription for the user and the recorded administration of the microneedle array patch and the one or more additional microneedle array patches.

9. A system, comprising:
a microneedle array patch including:
a base structure including first and second flat surfaces;
a plurality of microneedles disposed on the first flat surface of the base structure, the plurality of microneedles configured to extend out from the first flat surface, each microneedle of the plurality of microneedles including an agent that is loaded into at least a tip of the microneedle, the base structure configured to support the plurality of microneedles when force is applied against the second flat surface to insert the plurality of microneedles into skin; and
an adhesive disposed around the plurality of microneedles, the adhesive configured to secure the base structure and the plurality of microneedles to the skin for a predetermined period of time,
an identifier disposed on the microneedle array patch;
a sensor configured to read the identifier; and
a processor operatively coupled to the sensor, the processor configured to:
receive, from the sensor, the identifier of the microneedle array patch, wherein the identifier is associated with an identifier record;
query an information system to confirm the identifier and the microneedle array patch associated with the identifier are valid;
in response to receiving a response from the information system indicating that the identifier and the microneedle array patch are valid, present instructions to the user to administer the microneedle array patch; and
in response to receiving a response from the informing system indicating that the identifier and the microneedle array patch are not valid, present an alert to the user.

10. The system of claim 9, wherein the microneedle array is arranged on the first flat surface of the base structure in an area having a width of between about 2 mm and about 30 mm and a length of between about 10 mm and about 30 mm.

11. The system of claim 9, wherein the plurality of microneedles includes first and second sets of microneedles, the first set of microneedles having a first length and the second set of microneedles having a second length greater than the first length.

12. The system of claim 11, wherein the first length is in a range of between about 500 and about 800 μm and the second length is in a range of between about 50 μm and 250 μm.

13. The system of claim 9, wherein the plurality of microneedles are arranged on the first flat surface of the base structure in an array that is 10 microneedles to 50 microneedles by 10 microneedles to 50 microneedles.

14. The system of claim 9, wherein the processor is configured to query the information system to confirm the identifier and the microneedle array patch associated with the identifier are valid by sending the information system the identifier and information of the user such that the information system, in response to receiving the identifier and the information of the user, retrieves the identifier record associated with the identifier and a patient record associated with the user and determines that the identifier record and the patient record are associated with one another.

15. The system of claim 9, wherein the processor is further configured to:
obtain physiological information associated with the user measured by one of more sensors; and
send the physiological information to the information system such that the information system can monitor one or more vitals of the patient and provide feedback for adjusting usage of a subsequent microneedle array patch.

16. The system of claim 9, wherein the processor is further configured to:
record that the microneedle array patch and one or more additional microneedle array patches have been administered by the user; and
calculate a compliance score of the user based on a prescription for the user and the recorded administration of the microneedle array patch and the one or more additional microneedle array patches.

17. The system of claim 9, wherein the identifier is at least one of a quick-response (QR) code, a bar code, a radiofrequency identification (RFID) chip, or a serial number.

18. The system of claim 9, wherein the sensor is a first sensor, and the microneedle array patch further includes:
a second sensor configured to measure one or more physiological parameters; and
a transmitter configured to send the measurements of the one or more physiological parameters to the information system.

19. The system of claim 18, wherein in the information system is configured to:
process the measurements of the one or more physiological parameters to obtain treatment data including information associated with at least one of: confirmation of administration, efficacy of treatment, performance of the microneedle array patch, or side effects.

20. The system of claim 19, wherein the information system is further configured to send a notification to one or more third party devices based on the treatment data.

21. A method, comprising:
generating an identifier record for an identifier affixed to a microneedle array patch, the identifier being affixed to the microneedle array patch during manufacturing of the microneedle array patch, the microneedle array patch including a base structure and a plurality of microneedles disposed on a flat surface of the base structure, the base structure configured to provide support to the plurality of needles during insertion of the plurality of microneedles into skin;
generating an object record for the microneedle array patch;
associating, by the information system, the identifier record with the object record so that the microneedle array patch including the identifier affixed thereon is associated with the identifier
in response to receiving prescription information for a patient from a first device associated with a prescribing entity, associating a patient record of the patient with the identifier record so that the microneedle array patch including the identifier affixed thereon is associated with the patient;
receiving a request to validate the identifier and information of the patient from a second device associated with the patient prior to administration of the microneedle array patch;
validating the identifier and the information of the patient based on the object record, the identifier record, and the patient record and the associations therebetween, and sending a response to the second device based on the validation; and
sending, to a third device associated with managing electronic records of patients, treatment data associated with the administration of the microneedle array patch such that the third device can update electronic records of the patient.

22. The method of claim 21, wherein the plurality of microneedles includes first and second sets of microneedles, the first set of microneedles having a first length and the second set of microneedles having a second length greater than the first length.

23. The method of claim 21, wherein each microneedle of the plurality of microneedles includes a square obelisk structure.

24. The method of claim 21, wherein receiving the request to validate the identifier and the information of the patient includes:
receiving the request at a first layer of an information system, the first layer including an Application Programming Interface (API);
processing and routing the request from the first layer to one or more modules of a second layer for managing validation of the identifier and the information of the patient; and
validating the identifier and the information the patient at the second layer by retrieving information from a database of the information system.

25. The method of claim 24, further comprising:
receiving, at the first layer of the information system, the treatment data including the identifier, the prescription, and usage data of the microneedle array patch and one or more additional microneedle array patches;
processing and routing the treatment data to one or more modules of a second layer such that the treatment data is stored in the database of the information system and/or sent to the third device.

26. A method, comprising:
receiving, by a processor, an identifier of a microneedle array patch from a sensor, wherein the identifier is associated with an identifier record, wherein the microneedle array patch includes a base structure and a plurality of microneedles disposed on a flat surface of the base structure, the base structure configured to provide support to the plurality of needles during insertion of the plurality of microneedles into skin;
querying an information system to confirm the identifier and the microneedle array patch are valid;
in response to receiving a response from the information system indicating that the identifier and the microneedle array patch are valid, present instructions to the user to administer the microneedle array patch; and
in response to receiving a response from the informing system indicating that the identifier and the microneedle array patch are not valid, present an alert to the user.

27. The method of claim 26, wherein the plurality of microneedles includes first and second sets of microneedles, the first set of microneedles having a first length and the second set of microneedles having a second length greater than the first length.

28. The method of claim 20, wherein querying the information system to confirm the identifier and the microneedle array patch associated with the identifier are valid includes sending the information system the identifier and information of the user such that the information system, in response to receiving the identifier and the information of the user, retrieves the identifier record associated with the identifier and a patient record associated with the user and determines that the identifier record and the patient record are associated with one another.

29. The method of claim 26, further comprising:
obtaining physiological information associated with the user measured by one of more sensors; and
sending the physiological information to the information system such that the information system can monitor one or more vitals of the patient and provide feedback for adjusting usage of a subsequent microneedle array patch.

30. The method of claim 26, further comprising:
recording that the microneedle array patch and one or more additional microneedle array patches have been administered by the user; and
calculating a compliance score of the user based on a prescription for the user and the recorded administration of the microneedle array patch and the one or more additional microneedle array patches.
